(19) United States
(12) Patent Application Publication
(10) Pub. No.: US 2013/0216999 A1
(43) Pub. Date: Aug. 22, 2013

(54) METHODS AND KITS FOR THE DETECTION OF AN INFECTION IN SUBJECTS WITH LOW SPECIFIC ANTIBODY LEVELS

(71) Applicant: Tamar Jehuda-Cohen, Moshav Gimzo (IL)

(72) Inventor: Tamar Jehuda-Cohen, Moshav Gimzo (IL)

(73) Assignee: SMART Biotech Ltd., Rehovot, IL (US)

(21) Appl. No.: 13/821,029

(22) PCT Filed: Sep. 7, 2011
(86) PCT No.: PCT/IL11/00712

§ 371 Date: Apr. 23, 2013

Related U.S. Application Data
(60) Provisional application No. 61/380,617, filed on Sep. 7, 2010.

(51) Int. Cl. G01N 33/569

(52) U.S. Cl. CPC G01N 33/56983
USPC 435/5

(57) ABSTRACT

This invention relates to methods that enable the detection of antibodies against a latent infection, a chronic infection, a re-infection, and/or a breakthrough infection; enable the diagnosis of a latent infection, a chronic infection, a re-infection, and/or a breakthrough infection; and increase low anti-viral antibody levels, and a kit for the detection of virus-specific antibodies expressed at low levels.

### FIELD OF INVENTION

This invention relates to methods that enable the detection of antibodies against a latent infection, a chronic infection, a re-infection, and/or a breakthrough infection; enable the diagnosis of a latent infection, a chronic infection, a re-infection, and/or a breakthrough infection; and increase low anti-viral antibody levels, and a kit for the detection of virus-specific antibodies expressed at low levels.

### BACKGROUND OF THE INVENTION

Following chronic infection with certain pathogens (including viral or retroviral pathogens), the levels of antibodies against the pathogen are too low to be conclusively detected or may not be detectable at all. This phenomenon has been observed in some lung infections (e.g. pneumonia and tuberculosis), where the pathogen is localized to “inclusion bodies” which hide it from the immune system so that the antigenic stimulus is too low to maintain a long term measurable or detectable antibody production. In cases of retroviral infection, low antigenic stimulus could be attributed to a latent stage of the infection when the virus is maintained mainly as pro-viral DNA.

In some cases, upon infection or entry into the body, there is a measurable humoral immune response, which is not maintained for the whole duration of the infection. This issue is of concern for both diagnosis of infections which call for treatment (e.g. special antibiotics for certain types of pneumonia) and for the detection of the infection in cases of chronic infections.

Xenotropic murine leukemia virus (XMRV) is a virus that has “jumped” from mice to men. It has been recently implicated in prostate cancer and potentially in Chronic Fatigue Syndrome as well. The virus is sometimes, but not consistently, detected in the blood. XMRV may be infectious even at levels below the detection limit of currently available molecular assays. Therefore, the currently available molecular tests have very limited diagnostic capability.

Recent studies have shown that XMRV is present in approximately 2%-3% of healthy controls and 10% of immuno-compromised patients. This fact, coupled with the intermittent presence of the virus in the blood, has led to great concern regarding blood safety, and thus to an increased need to develop a diagnostic tool for those infected. Antibodies, the gold standard of diagnosis, are the tool of choice here, too. However, while antibody levels rise shortly after infection, enabling timely detection and diagnosis, their levels go down within a few months. Thus, the performance of any future diagnostic kit is hindered by very low levels of anti-XMRV antibodies in the infected individuals throughout most of the infection,

which seems to be chronic. Currently only a small percentage of those chronically infected with XMRV have detectable levels of antibodies in their blood. Increasing the analytical sensitivity of the assays being developed will not solve the diagnostic problem, as this increases the noise levels too, which will hinder the specificity of the detection. Thus, there is an unmet need to develop an antibody-based assay to enable detection of XMRV as well as other retroviruses and pathogenic infections in which the infection has a latent phase in which antibody levels are below or just at the level of detection. The invention provided herein addresses that need by providing a method of increasing the levels of the retrovirus- or pathogen-specific antibodies, thereby increasing the signal, while reducing the noise and bringing the diagnostic sensitivity of the assay to practical levels, making it feasible for both diagnostics and for introduction into the blood banks to increase the blood safety.

### SUMMARY OF THE INVENTION

In one embodiment, the present invention provides a method for detecting virus-specific antibodies in a sample from a subject with a chronic viral infection, a latent viral infection, a viral re-infection, a viral breakthrough infection, or a combination thereof comprising the steps of a) obtaining a whole blood sample; b) incubating the whole blood sample in a culture in the presence of a medium comprising an activator of (i) virus-activated lymphocytes, (ii) memory cells specific for said virus, (iii) virus-specific antibody expression, or (iv) a combination thereof; c) exposing the resultant culture of step b) to a viral antigen, thereby allowing an antigen-antibody immune complex to form; and d) detecting the antigen-antibody immune complex of step c), thereby detecting the presence of virus-specific antibodies.

In another embodiment, the present invention provides a method for diagnosing a chronic viral infection, a latent viral infection, a viral re-infection, a viral breakthrough infection, or a combination thereof in a subject comprising the steps of a) obtaining a whole blood sample; b) incubating the whole blood sample in a culture in the presence of a medium comprising an activator of (i) virus-activated lymphocytes, (ii) memory cells specific for said virus, (iii) virus-specific antibody expression, or (iv) a combination thereof; c) exposing the resultant culture of step b) to a viral antigen, thereby allowing an antigen-antibody immune complex to form; and d) detecting the antigen-antibody immune complex of step c), wherein the detection of said antigen-antibody immune complex indicates a viral infection in said subject.

In another embodiment, the present invention provides a method of increasing low anti-viral antibody levels in a whole blood sample to a detectable level comprising the steps of a) obtaining a whole blood sample; b) incubating the whole blood sample in a culture in the presence of a medium comprising an activator of (i) virus-activated lymphocytes, (ii) memory cells specific for said virus, (iii) virus-specific antibody expression, or (iv) a combination thereof; c) exposing the resultant culture of step b) to a viral antigen, thereby allowing an antigen-antibody immune complex to form; and d) detecting the antigen-antibody immune complex of step c), thereby increasing low anti-viral antibody levels to a detectable level.

In one embodiment, the invention provided herein relates to a kit for the detection of virus-specific antibodies expressed at low levels in a subject comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof of the whole blood sample selected from the group consisting of: a) an antibody against a lymphocyte cellular domain and b) a viral-derived peptide.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which:

FIG. 1 presents the antibody concentration in subjects with a chronic XMRV infection. “a” is a serum/plasma sample from a subject with XMRV antibody levels that are borderline positive, while “b” is a sample from a subject with XMRV antibody levels that are below the assay detection limit (i.e. cutoff ⋄). Squares and triangles indicate the XMRV antibody levels from untreated samples. Stars indicate the XMRV antibody levels of samples “a” and “b” when used in accordance with the methods of the present invention. Incubation of samples with an activator increased XMRV antibody levels in both samples to well over the cutoff of the detection level of the assay, thus enabling a positive true detection/diagnosis of XMRV infection.

### DETAILED DESCRIPTION OF THE INVENTION

In one embodiment, the present invention provides methods and kits for the detection of a viral infection in subjects with low specific antibody levels. In one embodiment, a chronic infection leads to decreased specific antibody levels. In one embodiment, a latent infection leads to decreased specific antibody levels.

In one embodiment, the present invention provides a method for detecting antibodies directed against a pathogen in a sample from a subject with an infection comprising the steps of a) obtaining a whole blood sample; b) incubating the whole blood sample in a culture in the presence of a medium comprising an activator of (i) a pathogen-activated lymphocyte, (ii) a memory cell specific for said pathogen, (iii) pathogen-specific antibody expression, or (iv) a combination thereof; c) exposing the resultant culture of step b) to a pathogen-specific antigen, thereby allowing an antigen-antibody immune complex to form; and d) detecting the antigen-antibody immune complex of step c), thereby detecting the presence of antibodies directed against a pathogen. In one embodiment, said infection is a chronic infection, a latent infection, a re-infection, a breakthrough infection, or a combination thereof.

In another embodiment, the present invention provides a method for diagnosing an infection in a subject comprising the steps of a) obtaining a whole blood sample; b) incubating the whole blood sample in a culture in the presence of a medium comprising an activator of (i) pathogen-activated lymphocytes, (ii) memory cells specific for said pathogen, (iii) pathogen-specific antibody expression, or (iv) a combination thereof; c) exposing the resultant culture of step b) to a pathogen-specific antigen, thereby allowing an antigen-antibody immune complex to form; and d) detecting the antigen-antibody immune complex of step c), wherein the detection of said antigen-antibody immune complex indicates an infection in said subject. In one embodiment, said infection is a chronic infection, a latent infection, a re-infection, a breakthrough infection, or a combination thereof.

In another embodiment, the present invention provides a method of increasing low anti-pathogen antibody levels in a whole blood sample to a detectable, or higher, level comprising the steps of a) obtaining a whole blood sample; b) incubating the whole blood sample in a culture in the presence of a medium comprising an activator of (i) pathogen-activated lymphocytes, (ii) memory cells specific for said pathogen, (iii) pathogen-specific antibody expression, or (iv) a combination thereof; c) exposing the resultant culture of step b) to a pathogen-specific antigen, thereby allowing an antigen-antibody immune complex to form; and d) detecting the antigen-antibody immune complex of step c), thereby increasing low anti-pathogen antibody levels to a detectable, or higher, level.

In one embodiment, the present invention provides a method for detecting antibodies directed against a pathogen in a sample from a subject with an infection comprising the steps of: a) incubating a whole blood sample from said subject in a culture in the presence of a medium comprising an activator of (i) a pathogen-activated lymphocyte, (ii) a memory cell specific for said pathogen, (iii) pathogen-specific antibody expression, or (iv) a combination thereof; b) exposing the resultant culture of step a) to a pathogen-specific antigen, thereby allowing an antigen-antibody immune complex to form; and c) detecting the antigen-antibody immune complex of step b), thereby detecting the presence of antibodies directed against a pathogen. In one embodiment, said infection is a chronic infection, a latent infection, a re-infection, a breakthrough infection, or a combination thereof.

In another embodiment, the present invention provides a method for diagnosing an infection in a subject comprising the steps of: a) incubating a whole blood sample from said subject in a culture in the presence of a medium comprising an activator of (i) a pathogen-activated lymphocyte, (ii) a memory cell specific for said pathogen, (iii) pathogen-specific antibody expression, or (iv) a combination thereof; b) exposing the resultant culture of step a) to a pathogen-specific antigen, thereby allowing an antigen-antibody immune complex to form; and c) detecting the antigen-antibody immune complex of step b), wherein the detection of said antigen-antibody immune complex indicates an infection in said subject. In one embodiment, said infection is a chronic infection, a latent infection, a re-infection, a breakthrough infection, or a combination thereof.

In another embodiment, the present invention provides a method of increasing low anti-pathogen antibody levels in a whole blood sample to a detectable, or higher, level comprising the steps of: a) incubating a whole blood sample from said subject in a culture in the presence of a medium comprising an activator of (i) a pathogen-activated lymphocyte, (ii) a memory cell specific for said pathogen, (iii) pathogen-specific antibody expression, or (iv) a combination thereof; b) exposing the resultant culture of step a) to a pathogen-specific antigen, thereby allowing an antigen-antibody immune complex to form; and c) detecting the antigen-antibody immune complex of step b), thereby increasing low anti-pathogen antibody levels to a detectable, or higher, level.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for the preparation of a composition for detecting antibodies directed against a pathogen in a whole blood sample from a subject with a chronic infection, a latent infection, a re-infection, a breakthrough infection, or a combination thereof In another embodiment, the infection is a chronic latent infection.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for the preparation of a composition for diagnosing an infection in a subject with a chronic infection, a latent infection, a re-infection, a breakthrough infection, or a combination thereof. In another embodiment, the infection is a chronic latent infection.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for the preparation of a composition for increasing low anti-pathogen antibody levels in a whole blood sample to a detectable, or higher, level in a subject with a chronic infection, a latent infection, a re-infection, a breakthrough infection, or a combination thereof In another embodiment, the infection is a chronic latent infection.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for detecting antibodies directed against a pathogen in a whole blood sample from a subject with a chronic infection, a latent infection, a re-infection, a breakthrough infection, or a combination thereof In another embodiment, the infection is a chronic latent infection.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for diagnosing an infection in a subject with a chronic infection, a latent infection, a re-infection, a breakthrough infection, or a combination thereof In another embodiment, the infection is a chronic latent infection.

In one embodiment, the present invention provides the use of a medium comprising an activator of (i) pathogen-activated lymphocytes, (ii) memory cells specific for said pathogen, (iii) pathogen-specific antibody expression, or (iv) a combination thereof for increasing low anti-pathogen antibody levels in a whole blood sample to a detectable, or higher, level in a subject with a chronic infection, a latent infection, a re-infection, a breakthrough infection, or a combination thereof. In another embodiment, the infection is a chronic latent infection.

In one embodiment, the medium described hereinabove further comprises a pathogen-specific antigen, thereby allowing an antigen-antibody immune complex to form. In another embodiment, the medium further comprises a means for detecting an antigen-antibody immune complex. In one embodiment, the pathogen is a virus. In another embodiment, the pathogen is a retrovirus. In another embodiment, the pathogen is a bacteria. In one embodiment, the pathogne, or infectious agent, is a microbe or microorganism such as a virus, bacterium, prion, or fungus that causes disease in its animal or plant host.

In one embodiment, the present invention provides a culture comprising a medium comprising an activator of memory cells as described hereinabove for the uses described hereinabove. In another embodiment, the present invention provides an activator of memory cells as described hereinabove for the uses described hereinabove. In another embodiment, the present invention provides a culture or medium or activator as described herein, a pathogen-specific antigen, and a means for detecting an antigen-antibody immune complex, such as, in one embodiment, an ELISA kit or other means known in the art or described herein.

In one embodiment, the present invention provides a kit for the enhancement of the levels of antibodies directed against an infection in a subject comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample, selected from the group consisting of: a) an antibody against a lymphocyte cellular domain; and b) a pathogen-derived peptide.

In another embodiment, the present invention provides a kit for detecting pathogen-specific antibodies in a subject comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample, selected from the group consisting of: a) an antibody against a lymphocyte cellular domain; and b) a pathogen-derived peptide.

In another embodiment, the present invention provides a kit for diagnosing an infection in a subject having a chronic infection, a latent infection, a pathogen re-infection, a breakthrough infection, or a combination thereof comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample, selected from the group consisting of: a) an antibody against a lymphocyte cellular domain; and b) a pathogen-derived peptide.

In another embodiment, the present invention provides a kit for increasing low anti-pathogen antibody levels in a whole blood sample to a detectable level comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample, selected from the group consisting of: a) an antibody against a lymphocyte cellular domain; and b) a pathogen-derived peptide.

In one embodiment, an infection as described herein is a chronic infection. In another embodiment, the infection is a latent infection. In another embodiment, the infection is a chronic latent infection. In another embodiment, the infection is a re-infection. In another embodiment, the infection is a breakthrough infection. In another embodiment, the infection is a chronic re-infection. In another embodiment, the infection is a latent reinfection. In another embodiment, the infection is a chronic breakthough infection. In another embodiment, the infection is a latent breakthrough infection. In another embodiment, the infection is a breakthrough re-infection. It is to be understood that blood samples from subjects with an infection by any combination of infection types are considered to be a part of the present invention.

In one embodiment, said infection is a chronic infection, a latent infection, a re-infection, a breakthrough infection, or a combination thereof.

In one embodiment, the present invention provides a method for detecting antibodies directed against a virus in a sample from a subject with a viral infection comprising the steps of a) obtaining a whole blood sample; b) incubating the whole blood sample in a culture in the presence of a medium comprising an activator of memory cells specific for said virus and the expression of virus-specific antibodies; c) exposing the resultant culture of step b) to a viral antigen, thereby allowing an antigen-antibody immune complex to form; and d) detecting the antigen-antibody immune complex of step c), thereby detecting the presence of virus-specific antibodies. In one embodiment, the infection is a chronic infection, a latent infection, a re-infection, a breakthrough infection, or a combination thereof.

In another embodiment, the present invention provides a method for diagnosing a viral infection in a subject comprising the steps of a) obtaining a whole blood sample; b) incubating the whole blood sample in a culture in the presence of a medium comprising an activator of (i) virus-activated lymphocytes, (ii) memory cells specific for said virus, (iii) virus-specific antibody expression, or (iv) a combination thereof; c) exposing the resultant culture of step b) to a viral antigen, thereby allowing an antigen-antibody immune complex to form; and d) detecting the antigen-antibody immune complex of step c), wherein the detection of said antigen-antibody immune complex indicates a viral infection in said subject. In one embodiment, said infection is a chronic infection, a latent infection, a re-infection, a breakthrough infection, or a combination thereof.

In another embodiment, the present invention provides a method of increasing low anti-viral antibody levels in a whole blood sample to a detectable level comprising the steps of a) obtaining a whole blood sample; b) incubating the whole blood sample in a culture in the presence of a medium comprising an activator of (i) virus-activated lymphocytes, (ii) memory cells specific for said virus, (iii) virus-specific antibody expression, or (iv) a combination thereof; c) exposing the resultant culture of step b) to a viral antigen, thereby allowing an antigen-antibody immune complex to form; and d) detecting the antigen-antibody immune complex of step c), thereby increasing low anti-viral antibody levels to a detectable level.

In one embodiment, the present invention provides a kit for the enhancement of the levels of antibodies directed against a viral infection from a subject comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof of the whole blood sample selected from the group consisting of: a) an antibody against a lymphocyte cellular domain; and b) a viral-derived peptide.

In another embodiment, the present invention provides a kit for detecting viral-specific antibodies in a subject comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof of the whole blood sample, selected from the group consisting of: a) an antibody against a lymphocyte cellular domain; and b) a viral-derived peptide.

In another embodiment, the present invention provides a kit for diagnosing an infection in a subject having a chronic viral infection, a latent viral infection, a viral re-infection, a viral breakthrough infection, or a combination thereof comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof of the whole blood sample, selected from the group consisting of: a) an antibody against a lymphocyte cellular domain; and b) a viral-derived peptide.

In another embodiment, the present invention provides a kit for increasing low anti-viral antibody levels in a whole blood sample to a detectable level comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof of the whole blood sample, selected from the group consisting of: a) an antibody against a lymphocyte cellular domain; and b) a viral-derived peptide.

In one embodiment, the present invention provides a method for detecting antibodies directed against a retrovirus in a sample from a subject with a retroviral infection comprising the steps of a) obtaining a whole blood sample; b) incubating the whole blood sample in a culture in the presence of a medium comprising an activator of (i) retrovirus-activated lymphocytes, (ii) memory cells specific for said retrovirus, (iii) retrovirus-specific antibody expression, or (iv) a combination thereof; c) exposing the resultant culture of step b) to a retroviral antigen, thereby allowing an antigen-antibody immune complex to form; and d) detecting the antigen-antibody immune complex of step c), thereby detecting the presence of retrovirus-specific antibodies. In one embodiment, said infection is a chronic infection, a latent infection, a re-infection, a breakthrough infection, or a combination thereof.

In another embodiment, the present invention provides a method for diagnosing a retroviral infection in a subject with a retroviral infection comprising the steps of a) obtaining a whole blood sample; b) incubating the whole blood sample in a culture in the presence of a medium comprising an activator of (i) retrovirus-activated lymphocytes, (ii) memory cells specific for said retrovirus, (iii) retrovirus-specific antibody expression, or (iv) a combination thereof; c) exposing the resultant culture of step b) to a retroviral antigen, thereby allowing an antigen-antibody immune complex to form; and d) detecting the antigen-antibody immune complex of step c), wherein the detection of said antigen-antibody immune complex indicates a retroviral infection in said subject. In one embodiment, said infection is a chronic infection, a latent infection, a re-infection, a breakthrough infection, or a combination thereof.

In another embodiment, the present invention provides a method of increasing low anti-retroviral antibody levels in a whole blood sample to a detectable level comprising the steps of a) obtaining a whole blood sample; b) incubating the whole blood sample in a culture in the presence of a medium comprising an activator of (i) retrovirus-activated lymphocytes, (ii) memory cells specific for said retrovirus, (iii) retrovirus-specific antibody expression, or (iv) a combination thereof; c) exposing the resultant culture of step b) to a retroviral antigen, thereby allowing an antigen-antibody immune complex to form; and d) detecting the antigen-antibody immune complex of step c), thereby increasing low anti-retroviral antibody levels to a detectable level.

In one embodiment, the present invention provides a kit for the enhancement of the levels of antibodies directed against a retroviral infection from a subject comprising: a container for retaining whole blood samples, wherein said container comprises a medium comprising one or more activators of (i) retrovirus-activated lymphocytes, (ii) memory cells specific for said retrovirus, (iii) retrovirus-specific antibody expression, or (iv) a combination thereof of the whole blood sample selected from the group consisting of: a) an antibody against a lymphocyte cellular domain; and b) a retroviral-derived peptide.

In another embodiment, the present invention provides a kit for detecting retroviral-specific antibodies in a subject comprising: a container for retaining whole blood samples, wherein said container comprises a medium comprising one or more activators of (i) retrovirus-activated lymphocytes, (ii) memory cells specific for said retrovirus, (iii) retrovirus-specific antibody expression, or (iv) a combination thereof of the whole blood sample, selected from the group consisting of: a) an antibody against a lymphocyte cellular domain; and b) a retroviral-derived peptide.

In another embodiment, the present invention provides a kit for diagnosing an infection in a subject having a chronic retroviral infection, latent retroviral infection, a retroviral re-infection, a breakthrough retroviral infection, or combination thereof comprising: a container for retaining whole blood samples, wherein said container comprises a medium comprising one or more activators of (i) retrovirus-activated lymphocytes, (ii) memory cells specific for said retrovirus, (iii) retrovirus-specific antibody expression, or (iv) a combination thereof of the whole blood sample, selected from the group consisting of: a) an antibody against a lymphocyte cellular domain; and b) a retroviral-derived peptide.

In another embodiment, the present invention provides a kit for increasing low anti-retroviral antibody levels in a whole blood sample to a detectable level comprising: a container for retaining whole blood samples, wherein said container comprises a medium comprising one or more activators of (i) retrovirus-activated lymphocytes, (ii) memory cells specific for said retrovirus, (iii) retrovirus-specific antibody expression, or (iv) a combination thereof of the whole blood sample, selected from the group consisting of: a) an antibody against a lymphocyte cellular domain; and b) a retroviral-derived peptide.

In one embodiment, the present invention provides a method for detecting antibodies directed against a Xenotropic murine leukemia virus (XMRV) in a sample from a subject with a XMRV infection comprising the steps of a) obtaining a whole blood sample; b) incubating the whole blood sample in a culture in the presence of a medium comprising an activator of (i) XMRV-activated lymphocytes, (ii) memory cells specific for XMRV, (iii) XMRV-specific antibody expression, or (iv) a combination thereof; c) exposing the resultant culture of step b) to a XMRV antigen, thereby allowing an antigen-antibody immune complex to form; and d) detecting the antigen-antibody immune complex of step c), thereby detecting the presence of XMRV specific antibodies. In one embodiment, said infection is a chronic infection, a latent infection, a re-infection, a breakthrough infection, or a combination thereof.

In another embodiment, the present invention provides a method for diagnosing a XMRV infection in a subject with a XMRV infection comprising the steps of a) obtaining a whole blood sample; b) incubating the whole blood sample in a culture in the presence of a medium comprising an activator of (i) XMRV-activated lymphocytes, (ii) memory cells specific for XMRV, (iii) XMRV-specific antibody expression, or (iv) a combination thereof; c) exposing the resultant culture of step b) to a XMRV antigen, thereby allowing an antigen-antibody immune complex to form; and d) detecting the antigen-antibody immune complex of step c), wherein the detection of said antigen-antibody immune complex indicates a XMRV infection in said subject. In one embodiment, said infection is a chronic infection, a latent infection, a re-infection, a breakthrough infection, or a combination thereof.

In another embodiment, the present invention provides a method of increasing low anti-XMRV antibody levels in a whole blood sample to a detectable level comprising the steps of a) obtaining a whole blood sample; b) incubating the whole blood sample in a culture in the presence of a medium comprising an activator of (i) XMRV-activated lymphocytes, (ii) memory cells specific for XMRV, (iii) XMRV-specific antibody expression, or (iv) a combination thereof; c) exposing the resultant culture of step b) to a XMRV antigen, thereby allowing an antigen-antibody immune complex to form; and d) detecting the antigen-antibody immune complex of step c), thereby increasing low anti-XMRV antibody levels to a detectable level.

In one embodiment, the present invention provides a kit for the enhancement of the levels of antibodies directed against a XMRV infection from a subject comprising: a container for retaining whole blood samples, wherein said container comprises a medium comprising one or more activators of (i) XMRV-activated lymphocytes, (ii) memory cells specific for XMRV, (iii) XMRV-specific antibody expression, or (iv) a combination thereof of the whole blood sample selected from the group consisting of: a) an antibody against a lymphocyte cellular domain; and b) an XMRV-derived peptide.

In another embodiment, the present invention provides a kit for detecting XMRV-specific antibodies in a subject comprising: a container for retaining whole blood samples, wherein said container comprises a medium comprising one or more activators of (i) XMRV-activated lymphocytes, (ii) memory cells specific for XMRV, (iii) XMRV-specific antibody expression, or (iv) a combination thereof of the whole blood sample, selected from the group consisting of: a) an antibody against a lymphocyte cellular domain; and b) a XMRV-derived peptide.

In another embodiment, the present invention provides a kit for diagnosing an infection in a subject having a chronic XMRV infection, a latent XMRV infection, a XMRV re-infection, a breakthrough XMRV infection, or a combination thereof comprising: a container for retaining whole blood samples, wherein said container comprises a medium comprising one or more activators of (i) XMRV-activated lymphocytes, (ii) memory cells specific for XMRV, (iii) XMRV-specific antibody expression, or (iv) a combination thereof of the whole blood sample, selected from the group consisting of: a) an antibody against a lymphocyte cellular domain; and b) a XMRV-derived peptide.

In another embodiment, the present invention provides a kit for increasing low anti-XMRV antibody levels in a whole blood sample to a detectable level comprising: a container for retaining whole blood samples, wherein said container comprises a medium comprising one or more activators of (i) XMRV-activated lymphocytes, (ii) memory cells specific for XMRV, (iii) XMRV-specific antibody expression, or (iv) a combination thereof of the whole blood sample, selected from the group consisting of: a) an antibody against a lymphocyte cellular domain; and b) a XMRV-derived peptide.

In accordance with the present invention, a blood sample is drawn into a test tube, which in one embodiment, is a vacuum-tube, a bottle, a well (as part of a multi well plate or as a single well/plate) or a flask, containing an effective concentration of a solution of a mitogen, such as, in one embodiment, pokeweed mitogen. The blood sample to be tested is cultured in vitro in the presence of the pokeweed mitogen. Other activators of human B cells may be used in place of or in addition to the pokeweed mitogen to achieve the same function. After incubation, an aliquot is taken from the top of the fluid and is then assayed for the presence of desired antibodies using standard ELISA procedures and/or Western Blot analysis and/or any other antibody detection system, which in one embodiment is a Rapid, Chemiluminescence, or chip system. If the sample is to be assayed at a later date, the blood may be centrifuged and the supernatant fluid may be collected, frozen and stored. Results may be verified utilizing the technique of polymerase chain reaction (PCR).

In one embodiment, the present invention increases antibody levels to “detectable” levels. In one embodiment, detectable levels of antibodies are levels that can be detected using antibody assays that are standard in the art. In one embodiment, detectable levels of antibodies are higher than the detection cut-off for an assay. In another embodiment, detectable levels are levels that are greater than 5% above the lower level of detection of a particular assay. In another embodiment, detectable levels are levels that are greater than 10% above the lower level of detection of a particular assay. In another embodiment, detectable levels are levels that are greater than 20% above the lower level of detection of a particular assay. In another embodiment, detectable levels are levels in which an assay can specifically detect antibody concentration without significant interference of non-specific, false positives (i.e. noise). In one embodiment, OD readings are used in conjunction with assays known in the art to measure antibody levels and/or concentration.

In accordance therewith, one related aspect of the invention is drawn to a method of diagnosis, which in one embodiment, is in vitro diagnosis, of the presence of a virus in a biological sample taken from a subject. In another related aspect, the biological sample is formed by a biological fluid, such as whole blood, cells, a tissue sample or biopsies of human origin. The term “sample” includes samples present in an individual as well as samples obtained or derived from the individual.

In one embodiment, the viral infection of the present invention is a latent viral infection. In another embodiment, the viral infection of the present invention is a chronic viral infection. In another embodiment, the viral infection of the present invention is a breakthrough viral infection. In another embodiment, the viral infection of the present invention is a viral re-infection. In another embodiment, the viral infection of the present invention is any combination of the above infections. In another embodiment, the viral infection of the present invention is both chronic and latent. In one embodiment, the viral infection of the present invention is chronic and/or latent. In one embodiment, “and/or” refers to either component or a combination thereof, such that a chronic and/or latent infection is a chronic infection, a latent infection, or both a chronic and latent infection.

In one embodiment, the initial viral infection results in the priming of naïve B cells and in the generation of plasma cells and memory B lymphocytes specific for a viral antigen that remain dormant and can be reactivated to produce antibody-secreting plasma cells that secrete antigen-specific antibodies. In another related aspect, the antibodies are specific for a viral antigen, protein, peptide, or epitope. In another related aspect, the antigen is a peptide, protein, sugar, component of the virus, etc.

In one embodiment, a viral infection related to the methods and kits of the present invention may be a persistent infection. In one embodiment, persistent infections are infections in which the virus is not cleared but remains in specific cells of infected individuals. Persistent infections often involve stages of both silent and productive infection without rapidly killing or even producing excessive damage of the host cells. In one embodiment, the persistent infection is a latent infection, a chronic infection, or a slow infection. In one embodiment, latent infection is characterized by the lack of demonstrable infectious virus between episodes of recurrent disease. In one embodiment, chronic infection is characterized by the continued presence of infectious virus following the primary infection and can include chronic or recurrent disease. In one embodiment, slow infection is characterized by a prolonged incubation period followed by progressive disease. Unlike latent and chronic infections, slow infection may not begin with an acute period of viral multiplication. During persistent infections, the viral genome can be either stably integrated into the cellular DNA or maintained episomally. In one embodiment, the persistent infection is human T-Cell leukemia virus, Epstein-Barr virus, cytomegalovirus, herpesvirus, varicella-zoster virus, measles, papovavirus, xenotropic murine leukemia virus-related virus (XMRV), prion, hepatitis virus, adenovirus, parvovirus or papillomavirus.

In one embodiment, a viral infection related to the methods and kits of the present invention may exhibit viral latency. In one embodiment, the viral latency is proviral latency and in another embodiment, the viral latency is episomal latency. In one embodiment, the virus is a herpesvirus, which in one embodiment, is varicella zoster virus, and, in another embodiment, is herpes simplex virus (HSV), which in one embodiment is HSV-1, and in another embodiment, HSV-2. In another embodiment, the virus is a gammaherpesvirinae, which in one embodiment is associated with episomal latency established in cells of the immune system. In one embodiment, the viral infection is Epstein-Barr Virus, which establishes episomal latency in B-cells. In another embodiment, the viral infection is cytomegalovirus (CMV), which in one embodiment, establishes latency within T lymphocytes, endothelial cells, and macrophages.

In one embodiment, a virus of the present invention is a retrovirus. In one embodiment, a retrovirus related to the methods and kits of the present invention is xenotropic murine leukemia virus (XMRV). In another embodiment, the retrovirus is hepatitis A virus (HAV). In another embodiment, the retrovirus is hepatitis B virus (HBV). In another embodiment, the retrovirus is hepatitis C virus (HCV). In another embodiment, the retrovirus is hepatitis D virus (HDV). In another embodiment, the retrovirus is hepatitis E virus (HEV). In another embodiment, the retrovirus is Human T-lymphotrophic virus-1 (HTLV-1). In another embodiment, the retrovirus is any combination of the retroviruses disclosed hereinabove. In another embodiment, the retrovirus is hepatitis A virus (HAV), hepatitis C virus (HCV), or hepatitis E (HEV) virus.

In one embodiment, the retrovirus is a Alpharetrovirus, which in one embodiment, is an Avian leukosis virus or Rous sarcoma virus. In another embodiment, the retroviral infection is a Betaretrovirus infection, which in one embodiment, is a Mouse mammary tumour virus or a human analogue thereof. In another embodiment, the retroviral infection is a Gammaretrovirus infection, which in one embodiment, is a Murine leukemia virus, Feline leukemia virus or a human analogue thereof. In another embodiment, the retroviral infection is a Deltaretrovirus infection, which in one embodiment, is a Bovine leukemia virus or a Human T-lymphotropic virus. In another embodiment, the retroviral infection is a Epsilonretrovirus infection, which in one embodiment, is a Walleye dermal sarcoma virus. In another embodiment, the retroviral infection is a Lentivirus infection, which in one embodiment, is a Human immunodeficiency virus 1, Simian immunodeficiency virus, or Feline immunodeficiency virus. In another embodiment, the retroviral infection is a Spumavirus infection, which in one embodiment, is a Simian foamy virus.

In another embodiment, the viral infection is a hepatitis A virus (HAV) infection, hepatitis B virus (HBV) infection, hepatitis C virus infection (HCV), hepatitis D virus (HDV) infection, or hepatitis E virus (HEV) infection. In another embodiment, the viral infection is a hepatitis A virus (HAV) infection, hepatitis C virus infection (HCV), or hepatitis E virus (HEV) infection.

In another embodiment, the pathogenic agent is any pathogenic agent, including a virus or retrovirus, known in the art to undergo a “hidden” or latent phase and/or a chronic phase. In one embodiment, the virus has low immunogenicity. In one embodiment, the infected subject is immune suppressed, which in one embodiment, is a mild immunosuppression.

In one embodiment, the methods of the present invention are for detecting antibodies in and for diagnosing subjects with a chronic viral infection, a latent viral infection, a viral re-infection, a viral breakthrough infection, or a combination thereof. In another embodiment, the methods of the present invention are for detecting antibodies in and for diagnosing subjects who are seropositive for the viral infection. In another embodiment, the methods of the present invention are for detecting antibodies in and for diagnosing subjects who have undergone seroconversion.

In one embodiment, virus latency (or viral latency) is the ability of a pathogenic virus to lie dormant within a cell, denoted as the lysogenic part of the viral life cycle. A latent viral infection is a type of persistent viral infection. A latent infection is a phase in the life cycle of certain viruses in which after initial infection, virus production ceases. However, the virus genome is not fully eradicated. The result of this is that the virus can reactivate and begin producing large amounts of viral progeny (the lytic part of the viral life cycle) without the host being infected by a new virus. The virus may stay within the host indefinitely. In one embodiment, virus latency is not identical to clinical latency, in which the virus is undergoing an incubation period but is not dormant.

In one embodiment, a chronic infection refers to a persistent and lasting infection. In one embodiment, the infection lasts more than three months. In another embodiment, the infection lasts more than six months. In another embodiment, the infection last more than nine months. In another embodiment, the infection lasts more than one year.

In one embodiment, a re-infection as described herein, is a second or subsequent infection by the same agent as a first infection. In one embodiment, the re-infection is with HIV. In another embodiment, the re-infection is with HCV. In another embodiment, the re-infection is with HBV. In one embodiment, the re-infection is with a different strain of the pathogen or virus. In one embodiment, the infectious agent is a retrovirus, and the re-infection is with a mutant strain of the same retrovirus, and in one embodiment, drug-resistant strain of the retrovirus. In one embodiment, an infection is a super-infection.

In one embodiment, a breakthrough infection as described herein, is an infection that occurred in a subject after vaccination of said subject with a vaccine against the infectious agent. In one embodiment, the vaccination was not a live virus vaccine. In one embodiment, the vaccination was a protein subunit vaccine. In one embodiment, the vaccination was a killed vaccine. In one embodiment, the vaccination was a viral mutant vaccine. In another embodiment, the vaccination was any type known in the art. In another embodiment, a breakthrough infection is an infection caused by the shedding of live virus from a vaccine. In one embodiment, a breakthrough infection is a full-blown infection. In another embodiment, a breakthrough infection is mild. In one embodiment, a breakthrough infection may cause significant or serious illness in a person with a compromised immune system, such as, in one embodiment, a subject infected with HIV.

In one related aspect, the culture resulting from the method provided herein results in a supernatant, and the supernatant is exposed to a viral antigen, thereby allowing an antigen-antibody immune complex to form. In one embodiment, culture medium refers to any medium that can be used to practice the present invention, including but not limited to RPMI 1640, with or without supplements such as antibiotics, glutamine, BSA, animal serum, cytokines, and/or lymphokines. Other culture media which may be used in practicing the present invention include, but are not limited to, Eagles, Dulbecco's, McCoy's, Media 199 and Waymouth's media.

In one embodiment, the terms “antibody” and “immunoglobulin” are used interchangeably herein. These terms are well understood by those in the field, and refer to a glycosylated (comprising sugar moieties) protein consisting of one or more polypeptides that specifically binds an antigen. The basic structural unit of an antibody is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The term “antibody” also includes any protein or peptide-containing molecule that comprises at least a portion of an immunoglobulin molecule, such as, but not limited to, one complementarity determining region (CDR) of a heavy chain or light chain constant region, a framework region, or any portion thereof. Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids) similarly comprise a variable region (of about 116 amino acids) and one of the aforementioned heavy chain constant regions or classes, e.g., gamma (of about 330 amino acids). The subunit structures and three-dimensional configurations of different classes of immunoglobulins are well known.

In one embodiment, methods and compositions of the present invention comprise anti-IgD, anti-IgG, anti-IgA, anti-IgE, or anti-CD19 antibodies. Methods of making these antibodies are well known in the art and the antibodies are commercially available.

In one embodiment, an “activator” for use in the compositions and methods of the present invention is a substance that induces the activation of memory cells. In another embodiment, an activator induces the activation specific lymphocytes. In another embodiment, an activator induces specific antibody expression. In another embodiment, an activator does a combination of the above. In one embodiment, the substance is a protein, while in another embodiment, it is a peptide, a nucleic acid molecule, a glycoprotein, etc. In one embodiment, “activation” of cells comprises inducing proliferation of cells, differentiation of cells, enhancement of cellular activity (in one embodiment, antibody production), secretion of various lymphokines and/or cytokines, or a combination thereof.

In one embodiment, the activator is a mitogen. In one embodiment, a “mitogen” is a chemical substance, or a mixture of substances, in one embodiment, a protein, that encourages a cell to commence cell division, triggering mitosis. In one embodiment, a mitogen triggers signal transduction pathways in which mitogen-activated protein kinase is involved, leading to mitosis. In one embodiment, mitogens of the present invention are used to induce mitosis and/or activation in memory B cells. In one embodiment, mitogens of the present invention are used to induce the formation of plasma cells from primed differentiating B cells and/or memory B cells.

In one embodiment, the mitogen of the compositions and methods of the present invention induces the activation of memory cells specific for the virus of interest. In another embodiment, the mitogen of the present invention induces the expression of viral-specific antibodies. In another embodiment, the mitogen of the present invention induces the transfer from memory cells to plasma cells.

In one embodiment, viral antigens are used in conjunction with mitogens to induce activation of memory B cells. Thus, in one embodiment, the compositions of the present invention, including those for use in the methods of the present invention, additionally comprise an antigen that is specific to the virus of interest which, in one embodiment, aids or enhances the transfer from memory cells to plasma cells. Similarly, the methods of the present invention may comprise incubating a blood sample in a medium comprising a mitogen and a viral antigen.

In one related aspect, the mitogen used in the invention provided herein can be either T-cell dependent or T-cell independent. In one embodiment, the mitogen used in the compositions and methods of the present invention acts on T-cells, B-cells, or both T cells and B cells. In one related aspect, the mitogen used to induce activation of memory B cells and the expression of virus-specific antibodies is pokeweed mitogen, which in one embodiment, stimulates both B- and T-cells. Other mitogens can be used in practicing the present invention and include, but are not limited to, lectins, such as, concanavalin A, which in one embodiment acts on T cells; bacterial endotoxins, which in one embodiment, is lipopolysaccharide (LPS), which in one embodiment, acts on B cells. In another embodiment, the mitogen is phytohaemagglutinin (PHA), which in one embodiment, acts on T cells. In another embodiment, the mitogen is leucoagglutinin (PHA-L), while in another embodiment, the mitogen is Pisum sativum agglutinin (PSA).

In another embodiment, the activator used in the composition and methods of the present invention is a cytokine, which in one embodiment is a signaling molecule secreted by specific cells of the immune system and glial cells. In one embodiment, said cytokine is an interleukin or interferon. In one embodiment, the cytokine is a lymphokine. In one embodiment, said lymphokine is Interleukin 1, Interleukin 2, Interleukin 3, Interleukin 4, Interleukin 5, Interleukin 6, Interleukin 10, Interleukin 12, Granulocyte-macrophage colony-stimulating factor, Interferon-gamma, or a combination thereof.

In one embodiment, the cytokine is a mediator of adaptive immunity. In another embodiment, the cytokine is a mediator of natural immunity. In another embodiment, the cytokine is tumor necrosis factor (TNF)-α, a type I interferon (IFN) (which, in one embodiment, is IFN-α or IFN-β), or a chemokine. In another embodiment, the cytokine is ransforming growth factor (TGF)-β. In another embodiment, the cytokine is a stimulator of hematopoesis, which in one embodiment, is Granulocyte-Macrophage Colony Stimulating Factor (GM-CSF), Macrophage colony-stimulating factor (M-CSF), or Granulocyte colony-stimulating facto (G-CSF). In another embodiment, the cytokine is Interleukin-17.

In another embodiment, the activator used in the compositions and methods of the present invention is a bacterially derived lipid A, a viral-derived peptide, a virus, a biological agent, an anti-immunoglobulin reagent, an antibody against a B-lymphocyte cellular domain, or a combination thereof.

In one embodiment, lipid A is a lipid component of an endotoxin held responsible for toxicity of Gram-negative bacteria and a very potent stimulant of the immune system, in one embodiment, activating monocytes or macrophages. In one embodiment, the lipid A has 6 acyl chains. In one embodiment, lipid A is from group of Gram-negative bacteria, including Escherichia coli (E. coli), Salmonella, Shigella, other Enterobacteriaceae, Pseudomonas, Moraxella, Helicobacter, Stenotrophomonas, Bdellovibrio, acetic acid bacteria, Legionella, cyanobacteria, spirochaetes, green sulfur or green non-sulfur bacteria.

In one embodiment, the activator used in the compositions and methods of the present invention is a lectin. In one embodiment, the lectin is a Mannose binding lectin, which in one embodiment, is Concanavalin A, Lentil lectin, or Snowdrop lectin. In another embodiment, the lectin is a Galactose/N-acetylgalactosamine binding lectin, which in one embodiment, is Ricin, Ricinus communis Agglutinin, RCA120; Peanut agglutinin; Jacalin; or Hairy vetch lectin. In another embodiment, the lectin is a N-acetylglucosamine binding lectin, which in one embodiment, is Wheat Germ agglutinin. In another embodiment, the lectin is a N-acetylneuraminic acid binding lectin, which in one embodiment, is Elderberry lectin, Maackia amurensis leukoagglutinin, or Maackia amurensis hemoagglutinin. In another embodiment, the lectin is a Fucose binding lectin, which in one embodiment is Ulex europaeus agglutinin and, in another embodiment, Aleuria aurantia lectin.

In another embodiment, a commercially available SMARTube™ comprising an activator is used as the activator in the compositions and methods of the present invention.

The optimal concentration of mitogen or other activator is easily determined without undue experimentation by one of ordinary skill in the art. In one related aspect, the mitogen concentration range used in the methods provided herein is between approximately 1:100 and 1:1600 dilutions of stock concentration. In another aspect, the concentration range is between 1:200 and 1:400 dilutions of stock. In another embodiment, the mitogen is a lectin, a bacterial endotoxin, a virus, lipid A or a lymphokine. In another aspect, the mitogen is a memory cell affecting mitogen, wherein the memory cell is a B-lymphocyte or a T-lymphocyte memory cell.

In one embodiment, the compositions of the present invention and the compositions for use in the methods of the present invention comprise a single activator, which in one embodiment is a mitogen, which in one embodiment, is pokeweed mitogen. In another embodiment, the compositions of the present invention and the compositions for use in the methods of the present invention comprise two activators, in another embodiment, three activators, in another embodiment, four activators, and, in another embodiment, five or more activators. In the case where more than one activator is included, each activator may be from a different class, or each activator may be from the same class. For example, the composition comprises a mitogen, a lymphokine, and a B-lymphocyte cellular domain antibody, which in one embodiment is pokeweed mitogen, interleukin-6, and anti-IgD antibody. In another embodiment, the composition comprises two mitogens, which in one embodiment are pokeweed mitogen and lipopolysaccharide. It is to be understood that any combination of activators as described herein are to be considered part of the invention.

In one related aspect, stimulation of memory cells is achieved by using antibodies against cellular membrane domains. In another embodiment, memory cells are stimulated by using antibodies against a B-lymphocyte cellular domain, which in one embodiment is a B-lymphocyte membrane domain. In one embodiment, the antibody is anti-IgD. In one embodiment, IgD is membrane-expressed by naïve B cells, initially primed B cells, and memory cells. In one embodiment, plasma cells do not express membrane IgD. In one embodiment, primed B cells that have not fully differentiated to plasma cells can be stimulated or activated by contacting them with anti-IgD.

In one embodiment, an “antibody” is a polypeptide ligand comprising at least a light chain or heavy chain immunoglobulin variable region which specifically recognizes and binds an epitope (e.g., an antigen, such as a tumor or viral antigen or a fragment thereof). This includes intact immunoglobulins and the variants and portions of them well known in the art, such as Fab′ fragments, F(ab)′.sub.2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3.sup.rd Ed., W.H. Freeman & Co., New York, 1997.

Typically, an immunoglobulin has a heavy and light chain. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs has been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V.sub.H CDR3 is located in the variable domain of the heavy chain of the antibody in which it is found, whereas a V.sub.L CDR1 is the CDR1 from the variable domain of the light chain of the antibody in which it is found.

References to “V.sub.H” or “VH” refer to the variable region of an immunoglobulin heavy chain, including that of an Fv, scFv, dsFv or Fab. References to “V.sub.L” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (e.g., see U.S. Pat. No. 5,585,089).

Thus, in one embodiment, an antibody against a B-lymphocyte membrane domain may be an antibody with the characteristics as described hereinabove and known in the art.

In another embodiment, the antibody is anti-IgM. In another embodiment, the antibody is directed against a B cell cellular domain (CD). In another embodiment, the antibody is directed against a T cell CD.

In one embodiment, the B-lymphocyte membrane domain is IgG, IgA, IgE, CD19, or any other membrane structure/domain known in the art. In another embodiment, the B-lymphocyte membrane domain is CD21 or CD81.

In one embodiment, the antibody class used to stimulate a memory cell includes, but is not limited to, an antibody from the IgG, IgD, IgA, or IgE class. In another aspect, the antibody is virus-specific. In another aspect, the antibody is XMRV-specific. In another aspect, stimulation of memory cells results in transformation of the memory cell to an antibody-secreting plasma cell, whereby the plasma cell secretes antigen-specific antibodies.

In one embodiment, a virus-activated lymphocyte of the present invention is a B-lymphocyte. In another embodiment, a virus-activated lymphocyte of the present invention is a T-lymphocyte. In one embodiment, a memory cell of the present invention is a B-lymphocyte. In another embodiment, a memory cell of the present invention is a T-lymphocyte. In one embodiment, a memory cell of the present invention is a memory B-lymphocyte. In another embodiment, a memory cell of the present invention is a memory T-lymphocyte.

In a related aspect, the B lymphocyte of the methods provided herein is a memory B-lymphocytic cell. In another related aspect, the T lymphocyte is a memory T-lymphocytic cell. In yet another related aspect, the activator provided herein activates a memory B-lymphocytic cell. In another embodiment, the activator activates a memory T-lymphocytic cell, and in another embodiment the activator activates both T and B cells.

In one embodiment, a viral-derived peptide for use in the compositions and methods of the present invention is an antigen. In another embodiment, the viral-derived peptide is an epitope, which in one embodiment, is an antigenic determinant. In another embodiment, the viral-derived peptide is immunogenic. In another embodiment, a viral-derived polypeptide may be used instead of a viral-derived peptide in the compositions and methods of the present invention.

In one embodiment, an antigen is a compound, composition, or substance that can stimulate the production of antibodies or a T cell response in a subject, including compositions that are injected or absorbed into the subject. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. An epitope typically includes at least 3, and more usually, at least 5, about 9, or about 8-10 amino acids in a unique spatial conformation.

In one embodiment, an XMRV-derived epitope is found within the amino acid sequence of NCBI Protein Database Ref. No. ACY30455-ACY30462 or ADF28703-ADF28719. In one embodiment, an XMRV-specific antigen is SU, TM, MA, CA, p12, NC, PR, RT, or IN. In one embodiment, an XMRV-specific antigen is an XMRV Gag polypeptide, an Env polypeptide, a Pol polypeptide, or a fragment thereof, or a polypeptide having at least about 80% sequence identity thereto. In one embodiment, an XMRV antibody detection system used in the methods and kits of the present invention are as described in Hohn et al. In Vitro. PLoS ONE 5(12): e15632. doi:10.1371/journal.pone.0015632, which is incorporated herein by reference.

In one embodiment, the methods of the present invention identify a hepatitis infection. According to this aspect and in one embodiment, the antigen used in the methods and kits of the present invention is a hepatitis surface antigen or a hepatitis core antigen.

In one embodiment, a pathogen- or virus-specific antigen is used in the compositions and methods of the present invention. In one embodiment, a pathogen-specific antigen is an immunogenic peptide, which in one embodiment, is a peptide which comprises an allele-specific motif or other sequence such that the peptide will bind an MHC molecule and induce a cytotoxic T lymphocyte (“CTL”) response, or a B cell response (e.g. antibody production or memory B cell proliferation) specific to the antigen from which the immunogenic peptide is derived.

In one embodiment, immunogenic peptides are identified using sequence motifs or other methods, such as neural net or polynomial determinations, known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Immunogenic peptides can also be identified by measuring their binding to a specific MHC protein (e.g. HLA-A02.01) and by their ability to stimulate CD4 and/or CD8 when presented in the context of the MHC protein.

In one related aspect, the present invention provides methods that enable an increase in the levels of the anti-viral antibodies, thus increasing the signal while reducing the noise. In another embodiment, the method of the present invention increases the levels of anti-viral antibodies without significantly increasing the noise levels. In another embodiment, the method of the present invention increases the levels of anti-viral antibodies without affecting the diagnosis (or diagnostic specificity).

In one embodiment, a complex between a sample XMRV antibody and a supplied XMRV antigen can detected through use of a probe directed against a serum antibody. The reaction mixture can be exposed to conditions sufficient for formation of a probe/antibody complex. The presence, absence or quantity of the probe, probe/antibody complex, or probe/antibody/antigen complex can then be detected. The probe can be directed against human immunoglobulin. For example, probes directed against human immunoglobulin include, but are not limited to, an antibody, an antigen-binding fragment thereof, an aptamer or an avimer. Detection of the presence, absence or quantity of a complex between a probe/antibody complex or probe/antibody/antigen complex can be according to any suitable means known in the art, as discussed herein. A probe for use with a sample XMRV antibody and a supplied XMRV antigen can include a label. Probe labels and detection thereof are known in the art.

In one embodiment, a competitive binding assay can be used to detect or quantify antibodies against an XMRV antigen or a gammaretrovirus antigen in a sample.

An XMRV antigen used to detect antibody that binds XMRV can be comprised by a eukaryotic cell ex vivo, such as a mammalian cell ex vivo or an insect cell ex vivo, or can be encoded by a polynucleotide and expressed in a microorganism, which can be a eukaryotic microorganism such as a yeast, or a prokaryotic microorganism such as an E. coli. A eukaryotic cell that expresses a polypeptide of the present teachings ex vivo can express the polypeptide on the cell surface or in the cytoplasm, or can secrete the polypeptide.

Some methods include providing at least one cell ex vivo that comprises an antigen that can be used to detect antibody against XMRV in a sample. The ex vivo cell can be a mammalian cell expressing a gammaretrovirus antigen, such as an XMRV antigen or an SFFV antigen ex vivo. The antigen can be, for example, an Env antigen of SFFV. Exemplary mammalian cells include a pro-B cell, such as a BaF3 cell a BaF3ER cell comprising an erythropoietin receptor. For example, a mammalian cell expressing at least one XMRV antigen can be a BaF3ER-SFFVEnV cell expressing Env protein of Friend spleen focus-forming virus (SFFV Env antigen).

In one embodiment, a solid surface or substrate can be used in conjunction with the above described antigen-based assay. Some methods of the present teachings include providing a solid support comprising an antigen that can be used to detect antibody against XMRV in a sample. A gammaretrovirus antigen, such as an XMRV antigen, can be immobilized on a solid support. A solid support can be, for example, a bead, a particle, or an ELISA plate, and an antigen can be adsorbed or attached to the support, e.g., through a covalent attachment. A cross-linking agent can be used to attach an XMRV antigen to a solid support. A sample of a subject can be introduced to a substrate such that an XMRV antibody, if present in the sample, binds to an XMRV antigen of the solid support.

In another embodiment, the present invention provides a kit for the enhancement of the levels of antibodies directed against an infection from a subject and thus the detection of said antibodies comprising: a container for retaining whole blood samples, wherein said container comprises a medium comprising two or more activators of (i) pathogen-activated lymphocytes, (ii) memory cells specific for said pathogen, (iii) pathogen-specific antibody expression, or (iv) a combination thereof of the whole blood sample selected from the group consisting of: a) a mitogen; b) an antibody against a lymphocyte cellular domain; and c) a pathogen-derived peptide. In one embodiment, the media comprises a mitogen and an antibody against a lymphocyte cellular domain. In another embodiment, the media comprises a mitogen and a pathogen-derived peptide. In one embodiment, the media comprises a mitogen and an antibody against a lymphocyte cellular domain. In another embodiment, the media comprises an antibody against a lymphocyte cellular domain and a pathogen-derived peptide. In another embodiment, the media comprises a mitogen, an antibody against a lymphocyte cellular domain; and a pathogen-derived peptide.

In another embodiment, the present invention provides a kit for the enhancement of the levels of antibodies directed against an infection from a subject and thus the detection of said antibodies comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample selected from the group consisting of: a) a mitogen; b) an antibody against a lymphocyte cellular domain; and c) a pathogen-derived peptide.

In another embodiment, the present invention provides a kit for the enhancement of the levels of antibodies directed against a pathogenic infection from a subject and thus the detection of said antibodies comprising: a container for retaining whole blood samples, wherein said container comprises a medium comprising one or more activators of (i) pathogen-activated lymphocytes, (ii) memory cells specific for said pathogen, (iii) pathogen-specific antibody expression, or (iv) a combination thereof of the whole blood sample selected from the group consisting of: a) an antibody against a lymphocyte cellular domain and b) a pathogen-derived peptide.

In another embodiment, the present invention provides a kit for detecting pathogen-specific antibodies in a whole blood sample from a subject with a chronic infection, a latent infection, a pathogen re-infection, a breakthrough infection, or a combination thereof comprising: a container for retaining a whole blood sample, wherein said container comprises a medium comprising an activator of (i) pathogen-activated lymphocytes, (ii) memory cells specific for said pathogen, (iii) pathogen-specific antibody expression, or (iv) a combination thereof, of said whole blood sample and instructions for use thereof. In one embodiment, the activator is a mitogen. In one embodiment, the activator is a pathogen-derived peptide. In one embodiment, the activator is an antibody against a lymphocyte cellular domain. In one embodiment, the infection is a viral infection. In one embodiment, the viral infection is a retroviral infection. In one embodiment, the viral infection is a hepatitis infection. In one embodiment, the viral infection is an XMRV infection. In one embodiment, the viral infection is an HIV infection. In another embodiment, the viral infection is any retroviral infection other than HIV.

In another embodiment, the present invention provides a kit for diagnosing a chronic infection, a latent infection, a pathogen re-infection, a breakthrough infection, or a combination thereof in a subject comprising: a container for retaining a whole blood sample, wherein said container comprises a medium comprising an activator of (i) pathogen-activated lymphocytes, (ii) memory cells specific for said pathogen, (iii) pathogen-specific antibody expression, or (iv) a combination thereof, of said whole blood sample and instructions for use thereof. In one embodiment, the activator is a mitogen. In one embodiment, the activator is a pathogen-derived peptide. In one embodiment, the activator is an antibody against a lymphocyte cellular domain. In one embodiment, the infection is a viral infection. In one embodiment, the viral infection is a retroviral infection. In one embodiment, the viral infection is a hepatitis infection. In one embodiment, the viral infection is an XMRV infection.

In another embodiment, the present invention provides a kit for increasing low anti-viral antibody levels in a whole blood sample from a subject with a chronic infection, a latent infection, a pathogen re-infection, a breakthrough infection, or a combination thereof to a detectable level comprising: a container for retaining a whole blood sample, wherein said container comprises a medium comprising an activator of (i) pathogen-activated lymphocytes, (ii) memory cells specific for said pathogen, (iii) pathogen-specific antibody expression, or (iv) a combination thereof, of said whole blood sample and instructions for use thereof In one embodiment, the activator is a mitogen. In one embodiment, the activator is a pathogen-derived peptide. In one embodiment, the activator is an antibody against a lymphocyte cellular domain. In one embodiment, the infection is a viral infection. In one embodiment, the viral infection is a retroviral infection. In one embodiment, the viral infection is a hepatitis infection. In one embodiment, the viral infection is an XMRV infection.

It is to be understood that other kits of the present invention may similarly comprise these components.

In one related aspect, the kit additionally comprises an assay for the detection of pathogen-specific antibodies. In one embodiment, the assay is in the kit separate from the container comprising the media comprising one or more or two or more activators, while in another embodiment, the assay is within the container comprising the media comprising one or more or two or more activators. According to this aspect and in one embodiment, a single container may conveniently be used for incubation of whole blood with the activator(s) and antibody detection, in one embodiment, providing a diagnosis at the end of the incubation period.

Thus, in one embodiment, the present invention provides a kit for the detection of antibodies directed against an infection from a subject, comprising: a container for retaining whole blood samples, wherein said container comprises:
1) a medium comprising two or more activators of (i) pathogen-activated lymphocytes, (ii) memory cells specific for said pathogen, (iii) pathogen-specific antibody expression, or (iv) a combination thereof of the whole blood sample selected from the group consisting of: a) a mitogen; b) an antibody against a lymphocyte cellular domain; and c) a pathogen-derived peptide; and
2) an assay for the detection of virus-specific antibodies.

In another embodiment, the present invention provides a kit for the detection of antibodies directed against an infection from a subject, comprising:
1) a container for retaining whole blood samples, wherein said container comprises: a medium comprising two or more activators of (i) pathogen-activated lymphocytes, (ii) memory cells specific for said pathogen, (iii) pathogen-specific antibody expression, or (iv) a combination thereof of the whole blood sample selected from the group consisting of: a) a mitogen; b) an antibody against a lymphocyte cellular domain; and c) a pathogen-derived peptide; and
2) an assay for the detection of virus-specific antibodies.

In one embodiment, the present invention provides a kit for the enhancement of the levels of antibodies directed against an infection from a subject and thus the detection of said antibodies. In another embodiment, the present invention provides a kit for the detection of antibodies directed against an infection from a subject. In another embodiment, the present invention provides a kit for diagnosing an infection in a subject with a chronic and/or latent infection.

In one embodiment, the media in a kit of the present invention comprises a mitogen and an antibody against a lymphocyte cellular domain. In another embodiment, the media comprises a mitogen and a pathogen-derived peptide. In one embodiment, the media comprises a mitogen and an antibody against a lymphocyte cellular domain. In another embodiment, the media comprises an antibody against a lymphocyte cellular domain and a pathogen-derived peptide. In another embodiment, the media comprises a mitogen, an antibody against a lymphocyte cellular domain; and a pathogen-derived peptide.

For use in diagnostic assays, the antibodies may optionally be associated with conventional labels which are capable, alone or in concert with other compositions or compounds, of providing a detectable signal. Where more than one antibody is employed in a diagnostic method, the labels are desirably interactive to produce a detectable signal. The label is detectable visually, e.g., colorimetrically, or by other known methods. A variety of enzyme and other systems have been described in the art which will operate to reveal a colorimetric signal in an assay. As one example, glucose oxidase (which uses glucose as a substrate) releases peroxide as a product. Peroxidase, which reacts with peroxide and a hydrogen donor such as tetramethyl benzidine (TMB), produces an oxidized TMB that is seen as a blue color. Other examples include horseradish peroxidase (HRP) or alkaline phosphatase (AP), and hexokinase in conjunction with glucose-6-phosphate dehydrogenase which reacts with ATP, glucose, and NAD+ to yield, among other products, NADH that is detected as increased absorbance at 340 nm wavelength. Other label systems that may be utilized in the methods of this invention are detectable by other means, e.g., colored latex microparticles [Bangs Laboratories, Ind.] in which a dye is embedded may be used in place of enzymes to form conjugates with the antibodies and provide a visual signal indicative of the presence of the resulting complex in applicable assays. Still other labels include fluorescent compounds, radioactive compounds or elements. Detectable labels for attachment to antibodies useful in diagnostic assays of this invention may be easily selected from among numerous compositions known and readily available to one skilled in the art of diagnostic assays. The methods and antibodies of this invention are not limited by the particular detectable label or label system employed. Suitably, these detectable systems may also be utilized in connection with diagnostic reagents composed of the peptides, proteins, and antibodies of the invention.

The biological sample collected by the methods provided herein may be brought into contact with, and immobilized onto, a solid phase support or carrier, such as nitrocellulose, polymer “beads”, chips, or other solid support or matrix, which is capable of immobilizing cells, cell particles, membranes, or soluble proteins. The support may then be washed with suitable buffers, followed by treatment with the detectably labeled anti-human antibody. The solid phase support may then be washed with buffer a second time to remove unbound antibody. The amount of bound label on the solid support may then be detected by conventional means.

In one embodiment, the container comprising the whole blood and the activators may further comprise a label in non-soluble form bound on a solid support, which in one embodiment, enables the subject to receive a diagnosis after an appropriate period of incubation. Such incubation periods are dependent on the label and on the time required for the activation step and are known in the art. In another embodiment, the label is in soluble form.

In one related aspect, methods of identifying antibodies produced by the methods provided herein through their binding affinities or specificities are very well known in the art and include methods such as immunoprecipitation or an in vitro binding assay, such as radioimmunoassay (RIA) or enzyme-linked immunoabsorbent assay (ELISA). Other well-known methods can be used to determine antibody binding affinities and these methods can be readily used, as will be understood by a skilled artisan. Polymerase chain reaction (PCR) technique may also be used to enhance the detection level of the preliminary incubation sample. It is to be understood that other assays known in the art to obtain or detect antibody interactions can also be used and these include but are not limited to immuno (western) blots, immunofluorescence assays, and the like. It should be understood by one of skill in the art that any number of conventional protein assay formats, particularly immunoassay formats, may be designed to utilize the isolated antigens and antibodies of this invention for the detection of anti-viral antibodies and for the detection of a viral infection in a subject. This invention is thus not limited by the selection of the particular assay format and encompasses assay formats which are known to those of skill in the art.

As a matter of convenience, a packaged combination of reagents in predetermined amounts with instructions for performing the diagnostic assay, e.g. kits, are also within the scope of the invention. The present invention includes a kit comprising a blood collection container containing an effective concentration of mitogen or other activator therein. The container can optionally contain a culture medium. In another related aspect, the container is a test tube or a flask. In yet another related aspect, the container is vacuum sealed. In one related aspect, the container is a vacutube. The blood collection container can be plastic, glass, silicon, synthetic membranes, or metal material (with or without special treatment or coating of the inner or outer surface), or any other material that is compatible with culturing blood. It is to be understood that the present invention also includes blood containing means other than a blood collection tube including, but not limited to, microtiter plates containing wells in which the blood can be incubated, tissue culture flasks, glass flasks such as an Erlenmeyer flask, and any other container in which the blood cells can be cultured.

In one embodiment, the container of the kit of the present invention is for retaining whole blood samples, or in another embodiment, holding, processing, storing, maintaining or collecting whole blood samples.

In another related aspect, the present invention's methods coupled with the present invention's kits enable the diagnostic sensitivity of the combined assay to be brought to practical levels, making it feasible for both diagnostics and for introduction into the blood banks to increase the blood safety.

Kits are also provided that are useful as a positive control for the diagnostic assays. For isolation and purification of anti-viral antibodies, the kit can contain viral proteins/antigens coupled to beads (e.g., sepharose beads or nanobeads or other nano-structures). Kits can be provided which contain the antibodies for detection and quantitation of anti-viral antibodies in vitro, e.g. in an ELISA or a Western blot. As with the article of manufacture, the kit comprises a container and a label or package insert on or associated with the container. The container holds a composition comprising at least one antigen recognized by the anti-viral antibodies. Additional containers may be included that contain, e.g., diluents and buffers, control antibodies. The label or package insert may provide a description of the composition as well as instructions for the intended in vitro or diagnostic use.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

The method of the present invention includes optionally separating the blood cells from the fluid portion of the blood so that the presence of antibodies, or the presence of antibody-producing cells can be determined. The separation of the blood cells from the fluid portion of the blood can be done by any of several methods well known to those of ordinary skill in the art, including centrifugation or filtration. In one embodiment, the blood cells are not physically separated from the fluid. In another embodiment, peripheral blood mononuclear cells (PBMCs), B-lymphocytes and T-lymphocytes may be separated from the blood prior to culture and assay. Methods of B cell and T cell enrichment are well known in the art and can be carried out by methods that include, but are not limited to, cell sorting/FACS. After incubation of the whole blood with the mitogen, fluid from the top of the blood can easily be extracted and tested for antibody. Optionally, the red blood cells can be lysed either by mild osmotic shock or with a mild detergent. In this way, the white blood cells remain viable.

Generally, the results of a test or assay according to the invention can be presented in any of a variety of formats. The results can be presented in a qualitative fashion. For example, the test report may indicate only whether or not a particular virus was detected, perhaps also with an indication of the limits of detection. The results may be presented in a semi-quantitative fashion. For example, various ranges may be defined, and the ranges may be assigned a score (e.g., 1+ to 4+) that provides a certain degree of quantitative information. Such a score may reflect various factors, e.g., the number of virus detected, the intensity of the signal (which may indicate the level of expression of virus bearing cells), etc. The results may be presented in a quantitative fashion, e.g., as a percentage of cells in which the virus is detected, as a viral protein concentration (as determined via antibody binding assay), etc. As will be appreciated by one of ordinary skill in the art, the type of output provided by a test will vary depending upon the technical limitations of the test and the biological significance associated with detection.

In one related aspect, the term “about” as refers to plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. In one embodiment, the subject is male. In another embodiment, the subject is female. In one embodiment, the subject is a pregnant or lactating female.

In one embodiment, xenotropic murine leukemia virus, xenotropic murine leukemia virus-related virus, XMRV, and XMLV are alternative names for the virus to be detected using the methods and compositions of the present invention. In one embodiment, XMRV belongs to the virus family Retroviridae and the genus gammaretrovirus. In one embodiment, XMRV has a single-stranded RNA genome that replicates through a DNA intermediate. In one embodiment, the genome of XMRV is approximately 8100 nucleotides in length, is 95% identical with several endogenous retroviruses of mice, and is 93-94% identical with several exogenous mouse viruses. In one embodiment, the sequences of several XMRV genomic sequences are almost identical, which in one embodiment is unusual in that retroviruses usually replicate their genomes with relatively low fidelity, leading to divergent viral sequences in a single host organism. Thus, in one embodiment, XMRV sequences are not likely to diverge significantly in a single host organism. Thus, in one embodiment, the methods of the present invention are practiced on a sample from a subject with a retroviral infection, which in one embodiment, replicates its genome with high fidelity.

It is to be understood that “retrovirus” may be replaced with “virus” or “pathogen” in the description of the methods and compositions of the present invention.

In one embodiment, “Stimmunology” is a device in which the methods of the present invention may be carried out conveniently and with minimum exposure of laboratory personnel to blood. In another embodiment, Stimmunology is a technology which overcomes low antibody levels and leads to antibody production in vitro, by cells which have been primed in vivo. In one embodiment, Stimmunology technology, is a pre-analytical step, in which specific antibodies are produced in vitro, enabling detection of infection by available antibody tests.

In one embodiment, the methods of the present invention may be practiced on tissue from a subject other than whole blood. In some embodiments, the sample is selected from the group consisting of a blood sample, a serum sample, a plasma sample, a cerebrospinal fluid sample, and a solid tissue sample. In some embodiments, the sample comprises cells selected from the group consisting of fibroblasts, endothelial cells, peripheral blood mononuclear cells, and haematopoietic cells, or a combination thereof. In one embodiment, lymph fluid, breast milk, mucus, saliva, and tears may be tested for antibodies to the pathogenic infection using the methods and compositions of the present invention.

In one embodiment, the present invention provides a method of determining the risk of a subject to develop prostate cancer comprising the step of determining if said subject has an XMRV infection using the methods of the present invention. In another embodiment, the present invention provides a method of treating, suppressing or inhibiting prostate cancer in a subject comprising the step of determining if said subject has an XMRV infection using the methods of the present invention and, if a subject is infected, treating said subject with retroviral integrase inhibitors; raltegravir (Merck & Co., brand name Isentress), L-000870812 (Merck & Co.), nucleoside reverse transcriptase inhibitors; tenofovir disoproxil fumarate (Gilead Sciences, brand name Viread), and zidovudine (GlaxoSmithKline, azidothymidine (AZT)), or a combination thereof.

In another embodiment, the present invention provides a method of determining the risk of a subject to develop chronic fatigue syndrome comprising the step of determining if said subject has an XMRV infection using the methods of the present invention. In another embodiment, the present invention provides a method of treating, suppressing or inhibiting chronic fatigue syndrome in a subject comprising the step of determining if said subject has an XMRV infection using the methods of the present invention and, if a subject is infected, treating said subject with retroviral integrase inhibitors; raltegravir (Merck & Co., brand name Isentress), L-000870812 (Merck & Co.), nucleoside reverse transcriptase inhibitors; tenofovir disoproxil fumarate (Gilead Sciences, brand name Viread), and zidovudine (GlaxoSmithKline, azidothymidine (AZT)), or a combination thereof.

In one embodiment, the present invention provides a method of determining the susceptibiltiy of a subject to cervical cancer comprising the step of determining if said subject has an human papillomavirus (HPV) infection using the methods of the present invention. In another embodiment, the present invention provides a method of treating, suppressing or inhibiting cervical cancer in a subject comprising the step of determining if said subject has an HPV infection using the methods of the present invention and, if a subject is infected, treating said subject for HPV using methods known in the art.

In one embodiment, treating the subject comprises administering one or more anti-retroviral agents for treating an XMRV-related disease or related disorder. For example, anti-retroviral agents can be used to treat an XMRV-related neuroimmune disease or an XMRV-related lymphoma. An anti-retroviral agent can be an anti-retroviral compound or pharmaceutical composition including an anti-retroviral compound. Examples of anti-retroviral agents that can be used to manage or treat an XMRV-related neuroimmune disease or an XMRV-related lymphoma include, but are not limited to, acyclovir, penciclovir (famciclovir), gancyclovir (ganciclovir), deoxyguanosine, foscarnet, idoxuridine, trifluorothymidine, vidarabine, sorivudine, zidovudine (AZT, ZVD, azidothyidine, e.g., Retrovir), didanosine (ddl, e.g., Videx and Videx EC), zalcitabine (ddC, dideoxycytidine, e.g., Hivid), lamivudine (3TC, e.g., Epivir), stavudine (d4T, e.g., Zerit and Zerit XR), abacavir (ABC, e.g., Ziagen), emtricitabine (FTC, e.g., Emtriva (formerly Coviracil)), entecavir (INN, e.g., Baraclude), apricitabine (ATC), tenofovir (tenofovir disoproxil fumarate, e.g., Viread), adefovir (bis-POM PMPA, e.g., Preveon and Hepsera), multinucleoside resistance A, multinucleoside resistance B, nevirapine (e.g., Viramune), delavirdine (e.g., Rescriptor), efavirenz (e.g., Sustiva and Stocrin), etravirine (e.g., Intelence), adefovir dipivoxil, indinavir, ritonavir (e.g., Norvir), saquinavir (e.g., Fortovase, Invirase), nelfinavir (e.g., Viracept), agenerase, lopinavir (e.g., Kaletra), atasanavir (e.g., Reyataz), fosamprenavir (e.g., Lexiva, Telzir), tipranavir (e.g., Aptivus), darunavir (e.g., Prezista), amprenavir, deoxycytosine triphosphate, lamivudine triphosphate, emticitabine triphosphate, adefovir diphosphate, penciclovir triphosphate, lobucavir triphosphate, amantadine, rimantadine, zanamiyir and oseltamivir, raltegravir (e.g., Isentress), elvitegravir (e.g., GS 9137 or JTK-303), MK-2048, maraviroc (e.g., Celsentri), enfuvirtide (e.g., Fuzeon), TNX-355, PRO 140, BMS-488043, plerixafor, epigallocatechin gallate, vicriviroc, aplaviroc, b12 (an antibody against HIV found in some long-term nonprogressors), griffithsin, DCM205, bevirimat, and vivecon. For example, one or more of AZT and cidofovir can be used to manage or treat an XMRV-related neuroimmune disease or an XMRV-related lymphoma. As another example, an interferon (e.g., interferon-β) can be used to manage or treat an XMRV-related neuroimmune disease or an XMRV-related lymphoma.

As another example, an agent for treating an XMRV-related disease or disorder can target the immune response to the retrovirus, such as NfkB inhibitors and monoclonal antibodies targeting virally infected cells including, but not limited to, Rituxan and Velcade.

In one embodiment, the present invention provides a method of determinining the risk of a subject to develop Chronic Fatigue Syndrome (CFS), fibromyalgia, Multiple Sclerosis (MS), Parkinson's Disease, Amyotrophic Lateral Sclerosis (ALS), Niemann-Pick Type C Disease, autism spectrum disorder (ASD), chronic lyme disease, or an XMRV-related lymphoma, which in one embodmient, is XMRV-related Mantle Cell Lymphoma (MCL) or a Chronic Lymphocytic Leukemia lymphoma (CLL). In another embodiment, the present invention provides a method of treating, suppressing or inhibiting the above diseases in a subject.

In another embodiment, the present invention provides a method of determining the susceptibiltiy of a subject to Chronic Fatigue Syndrome (CFS), fibromyalgia, Multiple Sclerosis (MS), Parkinson's Disease, Amyotrophic Lateral Sclerosis (ALS), Niemann-Pick Type C Disease, autism spectrum disorder (ASD), chronic lyme disease, or an XMRV-related lymphoma, which in one embodmient, is XMRV-related Mantle Cell Lymphoma (MCL) or a Chronic Lymphocytic Leukemia lymphoma (CLL).

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an anti-retroviral agent can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to reduce or eliminate XMRV replication or reduce or eliminate signs or symptoms of a neuroimmune disease or a lymphoma.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LDs₀ (the dose lethal to 50% of the population) and the ED₅0, (the dose therapeutically effective in 50% of the population). The dose ratio between toxic and therapeutic effects is the therapeutic index that can be expressed as the ratio LD₅₀IED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of an anti-retroviral agent can occur as a single event or over a time course of treatment. For example, an anti-retroviral agent can be administered daily, weekly, biweekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an XMRV-related neuroimmune disease or an XMRV-related lymphoma.

An anti-retroviral agent can be administered simultaneously or sequentially with another agent, such as an antibiotic, an antiinflammatory, or another agent. For example, an anti-retroviral agent can be administered simultaneously with another agent, such as an antibiotic or an antiinflammatory. Simultaneous administration can occur through administration of separate compositions, each containing one or more of an anti-retroviral agent, an antibiotic, an antiinflammatory, or another agent. Simultaneous administration can occur through administration of one composition containing two or more of an anti-vial agent, an antibiotic, an antiinflammatory, or another agent. An anti-retroviral agent can be administered sequentially with an antibiotic, an antiinflammatory, or another agent. For example, an anti-retroviral agent can be administered before or after administration of an antibiotic, an antiinflammatory, or another agent.

In one embodiment, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, any of the compositions for use in the present invention will comprise an activator of pathogen-activated lymphocytes and memory cells [active ingredient], in any form or embodiment as described herein. In some embodiments, any of the compositions of this invention will consist of an activator of pathogen-activated lymphocytes and memory cells [active ingredient], in any form or embodiment as described herein. In some embodiments, of the compositions of this invention will consist essentially of an activator of pathogen-activated lymphocytes and memory cells [active ingredient], in any form or embodiment as described herein. In some embodiments, the term “comprise” refers to the inclusion of the indicated active agent, such as the mitogen, viral-derived peptide, lectin, bacterial endotoxin, virus, lipid A, cytokine, or lymphokine or the mitogen and anti-IgD antibody, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic/diagnostic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In some embodiments, the methods of the present invention will comprise a step, in any embodiment as described herein. In some embodiments, any of the compositions of this invention will consist of a particular step, in any form or embodiment as described herein. In some embodiments, of the compositions of this invention will consist essentially of a particular step, in any form or embodiment as described herein. In some embodiments, the term “comprise” refers to the inclusion of the indicated step or steps, as well as inclusion of other steps as are known in the art. In some embodiments, the term “consisting essentially of refers to a method, whose only steps are the indicated steps, however, other steps may be included which are not involved directly in the diagnostic effect of the indicated steps. In some embodiments, the term “consisting” refers to a method, which contains only the indicated steps.

The following examples are presented in order to more fully illustrate the preferred embodiments of the invention. They should in no way be construed, however, as limiting the broad scope of the invention.

### EXAMPLES

### Example 1

### Activator Increases Specific Antibodies in Xenotropic Murine Leukemia Virus (XMRV)-Infected Seropositive Subjects

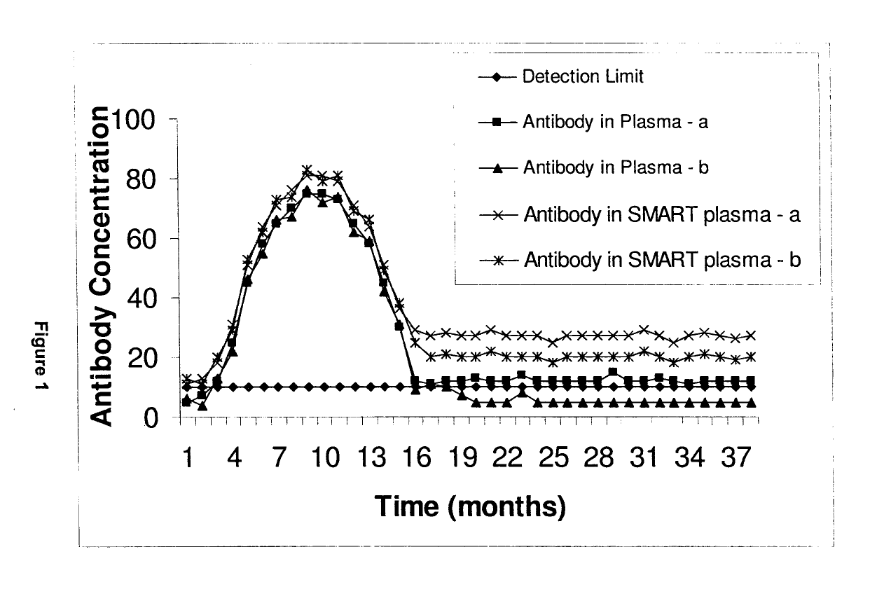

Blood from subjects known to be infected with XMRV is collected in anticoagulant, brought to the laboratory, and, within 24 h at room temperature, 1 ml of blood is transferred into a tube containing mitogen. The rest of the plasma is tested for XMRV antibodies (FIG. 1, solid lines) and kept for any additional future comparative testing. The blood is incubated in the tube containing mitogen for 3-5 days (5% CO₂, 37° C.), and the treated plasma is collected and tested for XMRV antibodies using the same kit/assay used to test the non-treated plasma.

The XMRV antibody concentration in the mitogen-treated plasma is higher than in the untreated plasma (Table 1). Thus, treated plasma can be diagnosed for XMRV infection with higher sensitivity.

TABLE 1
XMRV Antibody Concentrations (OD) in Untreated and Mitogen-
treated Plasma from XMRV-infected Subiects.

| | Plasma | |
|---|---|---|
| Sample # | (untreated) | Mitogen(s) |
| 1 | 0.110 | 0.300 |
| 2 | 0.200 | 0.550 |
| 3 | 0.101 | 0.200 |
| 4 | 0.090 | 0.140 |
| 5 | 0.087 | 0.170 |
| 6 | 0.130 | 0.560 |
| 7 | 0.400 | 0.450 |
| 8 | 0.070 | 0.095 |

Cutoff = 0.100

Blood samples from subjects not infected with XMRV is tested by the same protocol as a control group, showing that using the kit and methods of the present invention also increases the diagnostic specificity of the assay.

TABLE 2
XMRV Antibody Concentrations (OD) in Untreated and Mitogen-
treated Plasma from non-XMRV-infected Control Subjects

| | Plasma | |
|---|---|---|
| Sample # | (untreated) | Mitogen(s) |
| 1 | 0.030 | 0.023 |
| 2 | 0.099 | 0.015 |
| 3 | 0.010 | 0.012 |
| 4 | 0.090 | 0.014 |
| 5 | 0.012 | 0.017 |
| 6 | 0.013 | 0.005 |
| 7 | 0.024 | 0.025 |
| 8 | 0.007 | 0.009 |

Cutoff = 0.100

### Example 2

### Anti-IGD Further Increases Activator-Increased Specific Antibodies in Xenotropic Murine Leukemia Virus (XMRV)-Infected Seropositive Subjects

Blood samples are collected from individuals infected with XMRV, whose plasma antibody levels have gone down to at or below assay detection levels (e.g. OD readings close to cutoff). Aliquots of 1 ml of fresh, non-coagulated, whole blood (or PBMC) are incubated in anti-IgD, anti-IgD with mitogen(s), or mitogen(s) alone.

TABLE 3
OD Readings of Anti-XMRV Antibodies After Three Days of
Incubation with Anti-Igd, Mitogen(s), or Anti-Igd And Mitogen(s):

| | | | Anti-IgD | |
|---|---|---|---|---|
| | Plasma | | with | |
| Sample # | (untreated) | Anti-IgD | mitogen(s) | Mitogen(s) |
| 1 | 0.110 | 0.120 | 0.400 | 0.300 |
| 2 | 0.200 | 0.350 | 0.600 | 0.550 |
| 3 | 0.101 | 0.150 | 0.380 | 0.200 |
| 4 | 0.090 | 0.110 | 0.180 | 0.140 |
| 5 | 0.087 | 0.115 | 0.240 | 0.170 |
| 6 | 0.130 | 0.180 | 0.800 | 0.560 |
| 7 | 0.400 | 0.400 | 0.500 | 0.450 |
| 8 | 0.070 | 0.080 | 0.120 | 0.095 |

Cutoff OD - 0.100

Blood samples which were within the “grey zone” (i.e. close to the detection limit) of the assay give a positive reading following incubation with mitogen(s) and/or anti-IgD (Table 3). While mitogen(s) alone enables detection of XMRV specific antibodies in plasma, the addition of anti IgD improves antibody detection, especially in samples having a very low OD, and increases the specificity of the assay from 84% to 100%. Anti-IgD alone proves to be a relatively poor stimulant, providing a sensitivity of only 84% to the assay, which does not allow detection of many of the samples that were not treated with mitogen(s).

### Example 3

### Activator Increases Specific Antibodies in Hepatitis E Virus (HEV)-Infected Seropositive Subjects

Blood collected in anticoagulant from a local Beijing population was brought to the laboratory (within 24 h at RT) and 1 ml of blood was transferred into a container comprising activators (SMARTube™ available from http://www.smartube-bio.com/?CategoryID=190, accessed on Sep. 6, 2011). The rest of the plasma (untreated) was tested for HEV antibodies and kept for any additional future comparative testing. The blood in the container was incubated for 3-5 days (5% CO₂, 37° C.), and the plasma was collected and tested for HEV antibodies using the same kit/assay used to test non-treated blood samples.

TABLE 4
HEV Antibody Levels (ELISA) Following Stimulation of Whole
Blood with Activators

| | | |
|---|---|---|
| | | WT-HEV- |
| Beijing BB 12.04 | WT-HEV-P | ST |

| No. | sample code | Signal/CO | Signal/CO | ST/P |
|---|---|---|---|---|
| 1668 | 04222372 | 1.118 | 1.387 | 1.24 |
| 1682 | 04247337 | 1.133 | 1.410 | 1.24 |
| 1897 | 04220393 | 1.336 | 1.596 | 1.19 |
| 1973 | 04221904 | 2.559 | 2.740 | 1.07 |
| 1443 | 04192718 | 1.107 | 1.509 | 1.36 |
| 1211 | 04204895 | 1.027 | 1.859 | 1.81 |
| 1149 | 04220363 | 2.092 | 2.301 | 1.10 |

Table 4 represents the results of 7/15 seropositive samples, in which there was an increase in antibody levels following stimulation with activators. The actual level of antibody increase is several fold higher than expressed in the OD readings as is, and it can only be estimated, as the plasma is diluted 1:4. (1 ml of blood, which is ˜0.5 ml of plasma in 2 ml of media comprising activators), thus equal OD readings represent a four fold increase in antibody levels.

Sample 1211 is a good example of an OD reading right next to the cutoff which became a clear positive after stimulation with activators.

### Example 4

### Activator Increases Specific Antibodies in Hepatitis C Virus (HCV)-Infected Seropositive Subjects

Blood collected in anticoagulant, from a Hepatitis C virus (HCV) high risk population is brought to the laboratory (within 24 h, at room temperature), and 1 ml of blood is transferred into a container comprising activators (SMARTube™). The rest of the plasma (untreated) is tested for HCV antibodies and kept for any additional future comparative testing. The blood in the container is incubated for 3-5 days (5% CO₂, 37° C.) and the plasma is collected and tested for HCV antibodies using the same kit/assay used to test non-treated blood samples.

TABLE 5
HCV Antibody Levels (ELISA) Following Stimulation of Whole
Blood with an Activator

| | HCV Ab. Wantai | HCV Ab Wantai |
|---|---|---|
| | Plasma | SMARTplasma |
| | CO/0.140 | CO/0.14 |
| s2274 | 0.493 | 0.608 |
| s2011 | 1.611 | 2.149 |
| s2040 | 0.746 | 1.053 |
| s2046 | 0.78 | 1.051 |
| s2108 | 1.407 | 1.741 |
| s2125 | 1.264 | 1.966 |
| s2126 | 1.08 | 1.924 |
| s2135 | 0.949 | 2.008 |
| s2228 | 0.856 | 1.177 |

### Example 5

### Activator Increases Specific Antibodies in Hepatitis A Virus (HAV)-Infected Seropositive Subjects

Fourteen blood samples from healthy adult donors were pre-treated with five replicates of Activators (SMARTube™). prior to testing, using a commercially available test kit for HAV antibodies. Untreated and treated plasma were run in parallel, 100 μL from each, on the same plate according to the manufacturer's instruction for use.

There were two plasma positive samples with OD close to the cutoff which were also positive after the pre-treatment with Activators with marked increase in antibody levels, moving the results far from the cutoff.

The five replicates of blood from the same donor gave similar results, showing the reproducibility of the system. Results are presented as signal OD readings. All samples were run as pairs of untreated and treated plasma on the same ELISA plate, thus facilitating direct comparison.

The two plasma antibody positives, with very low levels of antibodies in the blood sample, had much higher levels of HAV antibodies after pre-treatment of the blood with mitogen, giving a more definitive HAV antibody positive result.

TABLE 6
HAV antibodies—table of results. (OD readings; Cutoff of 1.140)

| | | SMARTplasma | | | | |
|---|---|---|---|---|---|---|
| Sample # | Plasma | a | b | c | d | e |
| 6 | 1.362 | 2.468 | 2.480 | 2.332 | 2.387 | 2.504 |
| 18 | 1.297 | 2.062 | 2.196 | 2.149 | 2.132 | 2.117 |

The OD readings in the treated plasma in some seropositive individuals, and especially in those in the lower range of antibodies, are higher than in the untreated plasma—enabling diagnosis of infection with higher diagnostic sensitivity.

### Example 6

### An Activator Increases Specific Antibodies in Xenotropic Murine Leukemia Virus (XMRV)-Infected Seropositive Subjects

In vitro stimulation of XMRV antibody production in plasma before and after incubation with activator as described herein was tested. Monkeys were experimentally infected with XMRV, and then tested at different times post-infection for antibodies in the serum.

From the fresh blood collected, one ml was incubated in an experimental SMARTube™ for XMRV, for 5 days at 37° C. in a 5% CO₂ incubator. The rest of the plasma was collected and frozen until testing. After the incubation, the treated plasma (the supernatant fluid) was also collected and frozen away. Frozen paired samples (untreated plasma and treated plasma) were sent to be tested by a P 15E antigen XMRV antibody kit, while a separate pair of samples was tested using a p70 antigen XMRV antibody kit. The antibody levels were calculated using a known standard.

TABLE 7
P15E and p70 antibody levels in XMRV-infected monkeys

| | Subject ID | | | |
|---|---|---|---|---|
| Monkey data | PH0752 | RWy2 | PHw1 | PKd2 |
| Antibodies to - P15E | | | | |
| Plasma | 16705 | 189120 | 6532 | 358420 |
| SMARTplasma (diluted 1:4) | 4395 | 266506 | 2014 | 407497 |
| SMARTplasma after dilution | 21975 | 1332530 | 10070 | 2037485 |
| compensation (x5) | | | | |
| % increase in Ab by | 32 | 605 | 54 | 468 |
| SMARTube | | | | |
| Antibodies to p70 | | | | |
| Plasma | 31741 | 1172341 | 137797 | 1595971 |
| SMARTplasma (diluted 1:4) | 5877 | 1315635 | 29903 | 2080877 |
| SMARTplasma after dilution | 29385 | 6578175 | 149515 | 10404385 |
| compensation (x5) | | | | |
| % increase in Ab by | −7 | 461 | 9 | 552 |
| SMARTube | | | | |

There was an increase in antibody levels against P15E in blood samples from all four monkeys. In two of the subjects (RWy2 and PKd2), stimulation could be seen by the increase in antibody levels against P15E and p70 even with the plasma diluted 1:4 in the SMARTplasma.

### Example 7

### Activator Induced Increase in Specific Antibodies in Hepatitis B Virus (HBV)-Breakthrough Infection in Immunized Subjects

Blood samples from adult donors are pre-treated with activators prior to testing, using a commercially available test kit for HBV anti-core antibodies. Untreated and treated plasma are run in parallel, 75 μL from each, on the same plate according to the manufacturer's instruction for use.

Some of the blood donors are HBV-infected in spite of prior HBV immunization (immunization is currently with surface HBV antigen). The OD readings for anti-HBV-core in the plasma are below the level of detection of the assay. However, they are clear positive after the pre-treatment with activators; i.e. an increase in antibody levels moved the OD readings above the detection cutoff (The anti core antibodies are slow to rise to detectable and measurable levels following a true infection).

TABLE 8
Detection of HBV anti-core antibodies in mitogen-treated plasma
and untreated plasma from HBV-infected blood donors
(OD readings; Cutoff of 0.340)

| | Untreated | |
|---|---|---|
| Blood donors | Plasma | Treated Plasma |
| sample code | Signal/CO | Signal/CO |
| 02237 | 1.02 | 1.48 |
| 04733 | 0.97 | 1.71 |
| 02039 | 1.10 | 2.59 |
| 09271 | 0.07 | 1.50 |
| 00489 | 0.27 | 1.85 |
| 02036 | 0.60 | 2.30 |
| 02220 | 0.17 | 1.47 |

The OD readings in the treated plasma in individuals infected with HBV in spite of the immunization (=breakthrough infection), and especially in those in the lower range (or undetectable range) of antibodies in the untreated plasma, are higher—enabling diagnosis of infection with higher diagnostic sensitivity.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method for detecting virus-specific antibodies in a sample from a subject with a chronic viral infection, a latent viral infection, a viral re-infection, a viral breakthrough infection, or a combination thereof comprising:
a) incubating a whole blood sample from said subject in a culture in the presence of a medium comprising an activator of (i) virus-activated lymphocytes, (ii) memory cells specific for said virus, (iii) virus-specific antibody expression, or (iv) a combination thereof;
b) exposing the resultant culture of step a) to a viral antigen, thereby allowing an antigen-antibody immune complex to form; and
c) detecting the antigen-antibody immune complex of step b);
wherein said subject has low antibody levels or antibody levels at or below detection levels, thereby detecting the presence of viral-specific antibodies.

2-3. (canceled)

4. The method of claim 1, wherein said viral infection is a xenotropic murine leukemia virus (XMRV) infection, a hepatitis A virus (HAV) infection, hepatitis B virus (HBV) infection, hepatitis C virus infection (HCV), hepatitis D virus (HDV) infection, or hepatitis E virus (HEV) infection.

5. The method of claim 1, wherein the culture of step a) further comprises an antibody against a B-lymphocyte membrane domain.

6. The method of claim 5, wherein said antibody is anti-IgD or anti-IgM.

7. The method of claim 5, wherein said antibody is anti-IgG, anti-IgM, anti-IgA, anti-IgE, or anti-CD19.

8. The method of claim 1, wherein said activator is pokeweed mitogen.

9. The method of claim 1, wherein said activator is a viral-derived peptide, nucleic acid molecule, lectin, bacterial endotoxin, a virus, lipid A, a cytokine, or a lymphokine.

10. The method of claim 1, wherein said virus-activated lymphocytes or memory cells are B -lymphocytes.

11. The method of claim 1, wherein said virus-activated lymphocytes or memory cells are T-lymphocytes.

12. A method for diagnosing a chronic viral infection, a latent viral infection, a viral re-infection, a viral breakthrough infection, or a combination thereof in a subject comprising the steps of:
a) incubating a whole blood sample from said subject in a culture in the presence of a medium comprising an activator of (i) virus-activated lymphocytes, (ii) memory cells specific for said virus, (iii) virus-specific antibody expression, or (iv) a combination thereof;
b) exposing the resultant culture of step a) to a viral antigen, thereby allowing an antigen-antibody immune complex to form; and
c) detecting the antigen-antibody immune complex of step b);
wherein said subject has low antibody levels or antibody levels at or below detection levels and wherein the detection of said antigen-antibody immune complex indicates a viral infection in said subject.

13-22. (canceled)

23. A method of increasing low anti-viral antibody levels in a whole blood sample from a subject with a chronic viral infection, a latent viral infection, a viral re-infection, a viral breakthrough infection, or a combination thereof to a detectable level comprising:
a) incubating said whole blood sample in a culture in the presence of a medium comprising an activator of (i) virus-activated lymphocytes, (ii) memory cells specific for said virus, (iii) virus-specific antibody expression, or (iv) a combination thereof;
b) exposing the resultant culture of step a) to a viral antigen, thereby allowing an antigen-antibody immune complex to form; and
c) detecting the antigen-antibody immune complex of step b);
wherein said subject has low antibody levels or antibody levels at or below detection levels, thereby increasing low viral antibody levels to a detectable level.

24-33. (canceled)

34. A kit for the detection of virus-specific antibodies expressed at low levels in a subject comprising: a container for retaining whole blood samples, wherein said container comprises a medium comprising one or more activators of (i) virus-activated lymphocytes, (ii) memory cells specific for said virus, (iii) viral-specific antibody expression, or (iv) a combination thereof and further comprises a) an antibody against a lymphocyte cellular domain, b) a viral-derived peptide, c) a nucleic acid molecule, or d) a combination thereof.

35. The kit of claim 34, wherein said viral-derived peptide is a viral antigen.

36. The kit of claim 34, wherein said lymphocyte cellular domain is a T-lymphocyte membrane domain.

37. The kit of claim 34, wherein said lymphocyte cellular domain is a B-lymphocyte membrane domain.

38-39. (canceled)

40. The kit of claim 37, wherein said B-lymphocyte membrane domain is IgD, IgG, IgA, IgE, IgM, or CD19.

41. (canceled)

42. The kit of claim 34, wherein said activator is pokeweed mitogen, a viral-derived peptide, lectin, bacterial endotoxin, a virus, lipid A, a cytokine, or a lymphokine.

43-45. (canceled)

46. The kit of claim 34, wherein said kit additionally comprises an assay for the detection of viral-specific antibodies.

47. The kit of claim 46, wherein said assay is an enzyme linked immunosorbent assay, a western blot, or an immunofluorescence assay.

48-52. (canceled)

53. The method of claim 1, wherein said virus-activated lymphocytes or memory cells are both B-lymphocytes and T-lymphocytes.
